(19) United States
(12) Patent Application Publication
(10) Pub. No.: US 2013/0303825 A1
(43) Pub. Date: Nov. 14, 2013

(54) METHOD AND APPARATUS FOR PRODUCING IRRADIATION PLANNING

(71) Applicants: Christoph Bert, Uttenreuth (DE); Sebastian Hild, Muehlheim (DE)

(72) Inventors: Christoph Bert, Uttenreuth (DE); Sebastian Hild, Muehlheim (DE)

(73) Assignee: GSI Helmholtzzentrum Fuer Schwerionenforschung GmbH, Darmstadt (DE)

(21) Appl. No.: 13/977,938

(22) PCT Filed: Jan. 17, 2012
(86) PCT No.: PCT/EP12/50654

§ 371 Date: Jul. 2, 2013

(30) Foreign Application Priority Data
Jan. 18, 2011 (DE) 10 2011 000 204.9

(51) Int. Cl. A61N 5/10

(52) U.S. Cl. CPC A61N 5/1037
USPC 600/1

(57) ABSTRACT

A method for drawing up an irradiation plan includes at least one of calculating, assessing, displaying and taking into consideration effects of at least one uncertainty on the irradiation plan, at least one of at times and in certain areas.

### CROSS REFERENCE TO RELATED APPLICATIONS

This application is a U.S. National Phase application under 35 U.S.C. §371 of International Application No. PCT/EP2012/050654, filed on Jan. 17, 2012, and claims benefit to German Patent Application No. DE 10 2011 000 204.9, filed on Jan. 18, 2011. The International Application was published in German on Jul. 26, 2012, as WO 2012/098125 A1 under PCT Article 21 (2).

### FIELD

The invention relates to a method for drawing up an irradiation plan. Moreover, the invention relates to a device for drawing up an irradiation plan.

### BACKGROUND

Nowadays, particle beams are used in various realms of technology. In this context, all kinds of different types of particles are used, depending on the application purpose and on the available funds. Thus, for example, particle beams with photons, electrons, protons and heavy ions (e.g. helium ions, carbon ions, etc.), pions, mesons, etc. are used. Sometimes, mixtures of different types of particles are used as well. Depending on the type of particle and on the requisite energy, the accelerators needed to generate the particle beam are built in different ways and some of them are quite complex.

A technical field in which some particle beams have been used successfully for many years is in the realm of medical technology. Here, for example, photon radiation (especially X-ray radiation) has been used for a number of decades for cancer treatment.

Particularly in recent years, cancer therapy with heavy ion particle beams has started to gain a foothold as a permanent fixture in medical technology. A major advantage of particle beams with hadrons, especially heavy ions, is that they have a pronounced Bragg peak. This means that, as these specific particles pass along their path through matter, they do not release their kinetic energy uniformly to the tissue that is to be penetrated. Rather, most of the energy release of heavy ions is concentrated on a relatively short area, shortly before the particles “get stuck” in the tissue they have penetrated. This property makes it possible to systematically deposit a specific energy dose in a target volume area (especially also in the z-direction parallel to the particle beam) without the surrounding tissue regions (that is to say, for instance, the tissue regions in front of or behind the target region) being exposed to a (higher) dose. It is precisely this property that permits a highly effective cancer treatment that is gentle on the patient.

In modern-day therapy methods, scanning methods (especially raster scanning methods, including intensity-modulated raster scanning methods) are being used to an ever-greater extent. Here, a pencil-thin particle beam (a so-called pencil beam) is used to successively reach the tissue that is to be treated. A great advantage of such scanning methods is that tumors of almost any shape can be treated.

In actual practice, the therapy with heavy ion beams is carried out using a so-called “irradiation plan”. This is because there are numerous different interactions between the heavy ions of the particle beam and the tissue, which are very complicated to take into account by means of calculations. For example, even with today's high-speed computers, a numerical treatment of the problem still requires computation times ranging from minutes to hours.

At the beginning of a treatment, first of all, the physician prescribes a (biologically effective) dose distribution for the patient. Here, the dose distribution depends on the specific volume area in the body of the patient. To put it in simpler terms, the effective dose in the region of the tumor has to be above a damage limit value, so that the tumor tissue is destroyed. However, the surrounding tissue should only be exposed to the smallest extent possible (in the ideal case, not at all, although as a rule, this is technically not possible). Particularly when critical tissue regions such as, for instance, so-called OARs, short for “organ at risk”, are adjacent to the tumor tissue, an upper limit value is often specified here that must not be exceeded in order to ensure these critical tissue regions are not damaged. Such critical tissues can be, for instance, major blood vessels, nerve nodes or the spinal cord.

Based on the dose distribution prescribed by the physician, the irradiation plan is subsequently drawn up. In this process—roughly speaking—the (biologically effective) dose distribution prescribed by the physician is converted into a format (set of control parameters) that can be used by the radiation-generating device. In actual practice, this is done in that a calculation is made as to the biological effect caused by a thin particle beam that is introduced into the target volume area of the target body from one or more directions with a certain (three-dimensional) motion pattern (in the case of scanning methods). The biological effects thus calculated are compared to the biologically effective dose distribution prescribed by the physician. Optimization methods are carried out in an attempt to minimize the difference between the prescribed dose distribution and the biologically effective dose distribution introduced on the basis of the calculation.

The irradiation plan pays particular attention to the dose amounts that the particle beam introduces into other volume areas (for example, into individual raster points). Here, as a rule, the dose amounts behind (distal to) the “actual” volume area (raster point) are very small (so that they can often be ignored), whereas, as seen in the direction of the beam, quite relevant dose depositions can be made in front of (proximal to) the “actual” volume area (raster point). Moreover—especially in the case of heavy ion particle radiation—it must be taken into account that the so-called relative biological effectiveness (RBE) depends on physical parameters in a complex and non-linear manner. For example, the relationship between the deposited physical dose (corresponding to the energy loss of the particle beam) and the tissue damage (that is to say, the biologically effective dose) typically changes as a function of the particle energy. Moreover—once again, especially in the case of heavy ion particle radiation—so-called secondary radiation can occur due to decaying heavy ions. This is also associated with non-linear biological effects. Moreover, the deposited dose (the physically as well as biologically effective dose) changes, depending on the type of tissue so that bones, muscle tissue, blood vessels, cavities and the like (among others), have to be weighted differently within the scope of the irradiation planning An overview of the problems encountered when drawing up irradiation plans can be found, for instance, in the two articles “Treatment Planning for Heavy Ion Radiotherapy: Clinical Implementation and Application” by M. Krämer, O. Jäkel, G. Haberer, G. Kraft, D. Schardt and O. Weber in Phys. Med. Biol., Vol. 45, Year 2000, pages 3.299 to 3.317, as well as “Treatment Planning for Heavy Ion Radiotherapy: Calculation and Optimisation of Biologically Effective Dose” by M. Krämer and M. Scholz in Phys. Med. Biol., Vol. 45, Year 2000, pages 3.319 to 3.330.

A major problem encountered in the irradiation planning commonly carried out nowadays is that, as a rule, they are based on a fixed data set of parameters. Such parameters are, for example, the operating parameters of the accelerator, the tumor distribution, the distribution of the different types of tissues, the magnitude and energy of the particle beam, the position of the patient relative to the accelerator, the location of the tumor inside the patient, the beam profile, the movement of the patient as well as the movement of tumor regions due to breathing, heartbeat and other internal movements of the patient, etc. These parameters (each assumed to be fixed) are used to draw up the irradiation plan.

As is generally the case in the realm of technology, however, imprecisions are encountered here as well that can stem, for instance, from device fluctuations, measuring inaccuracies and the like. When it comes to drawing up irradiation plans, it has been found that the fluctuations of certain parameters can have very great effects on the resulting irradiation planning and on the effectively deposited biologically effective dose. Thus, the case could occur that an irradiation plan that theoretically generates an actually very good dose distribution is highly disadvantageous in actual practice, since it reacts very sensitively to just slight parameter fluctuations by undergoing major dose distribution changes (that is to say, it is not robust). Currently, a great deal of experience and “feel” on the part of the person (as a rule, a physician and/or a medical physicist) who is drawing up the irradiation plan go into assessing the “robustness” of an irradiation plan to withstand fluctuations of parameter values. A “genuine”, especially a quantitative assessment of the robustness of the irradiation plan, however, does not take place.

Such an assessment—if possible also quantitative—of the “robustness” of the irradiation plan, however, is desirable in order to be able to effectuate improved dose distributions and thus to ultimately achieve better therapy outcomes.

### SUMMARY

In an embodiment, the present invention provides a method for drawing up an irradiation plan. The method includes at least one of calculating, assessing, displaying and taking into consideration effects of at least one uncertainty on the irradiation plan, at least one of at times and in certain areas.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a schematic flow diagram for a method for drawing up an irradiation plan;

FIG. 2 shows a device for drawing up an irradiation plan, in a schematic perspective view;

FIG. 3 shows a first example of a display possibility of the effects of uncertainties on the irradiation plan;

FIG. 4 shows a second example of a display possibility of the effects of uncertainties on the irradiation plan.

FIGS. 5-8 show example displays of dose fluctuations.

### DETAILED DESCRIPTION

An aspect of the present invention is thus to provide a method for drawing up an irradiation plan that is improved in comparison to the state of the art. Another objective of the invention is to propose a device for drawing up an irradiation plan that is improved in comparison to the state of the art.

In an embodiment, the present invention provides a method for drawing up an irradiation plan in such a way that, at least at times and/or at least in certain areas, the effects of at least one uncertainty on the irradiation planning are calculated, assessed, displayed and/or taken into consideration. In this manner, the uncertainty that can arise due to erroneous assumptions, measuring errors or parameter fluctuations of the accelerator, of the measuring sensor systems or of the patient (etc.) that are unavoidable in actual practice can be ascertained at least qualitatively but preferably also quantitatively. Preferably, the uncertainty is taken into consideration automatically. In particular, as a rule, based on nominal parameter values, certain fluctuations or fluctuation patterns can be impressed onto a particular “starting value”. The magnitude of the impressed fluctuations or rather, the type of fluctuation patterns, is determined here on the basis of fluctuations that actually occur or fluctuations that can be realistically expected. Moreover, an automated approach does not rule out that (especially up to a certain degree) manual action can be carried out or that a manual specification can be used to select different automated fluctuation patterns. Very generally speaking, within the scope of the proposed method, such a (partially) automated consideration of at least one uncertainty can prove to be advantageous, especially with respect to the possible refinements of the proposed method presented below. Consequently, the result of the irradiation planning process can become better and, in particular, more robust. An other advantage is that, as a rule, advantageous irradiation plans can end up being much less dependent on the ability, amount of experience, “feel”, etc. of the person(s) involved in the irradiation planning As a result, for example, the irradiation planning can be drawn up by technical personnel that is less highly qualified than the persons involved nowadays. The effects that one uncertainty (or several uncertainties, especially a large number of relevant uncertainties, particularly essentially all and/or all relevant uncertainties) has on the results of the irradiation planning can be calculated, assessed, displayed and/or taken into consideration in any desired manner. A calculation can be carried out, for example, in such a way that the values are merely calculated internally. However, it is more useful if something is, in fact “done” with the calculated values. In particular, it can prove to be useful if an assessment of the (preliminary) irradiation planning, especially an assessment of the (preliminary) irradiation planning drawn up by the irradiation planning device itself, is carried out. For instance, this can done in that an irradiation plan is blocked or not output if the effects of an uncertainty are too great and especially if they lie above a certain limit value. Furthermore, a “permissible” irradiation plan can be drawn up and/or released that especially lies below a certain limit value. However, it is also advantageous if the effects of the uncertainties are displayed to the person or persons drawing up the irradiation plan, for example, at least at times and/or at least partially qualitatively and/or quantitatively. The persons involved can then (for instance, based on their experience) optimize the irradiation plan in such a way that the effects of the uncertainties are, for example, particularly small and/or are advantageous in some other manner (in other words, they are especially robust). However, it can also be especially advantageous if the effects of the at least one uncertainty are taken into account (automatically) at least at times and/or at least partially within the scope of the irradiation planning Thus, for example, an optimization algorithm can autonomously perform an optimization, also in terms of the effects of the at least one uncertainty (in other words, an optimization in terms of the robustness of the irradiation plan), so that, as a result, for example, a (local) minimum can be reached. The term “effects of an uncertainty” refers especially to a fluctuation of the dose distribution in the volume that is to be irradiated or in parts of the volume that is to be irradiated (or that is not to be irradiated). In particular, this can refer to under-dosing (which is to be avoided to the extent possible) in the region of the tumor that is to be treated and/or to overdosing in the healthy tissue, especially in regions where sensitive tissues (such as OARs) are present.

It is advantageous if, with this method, the at least one uncertainty, at least at times and/or at least partially, constitutes a fluctuation of at least one parameter, especially a fluctuation of at least one parameter within a typical and/or maximally expected scope. In this context as well, an automated consideration of the uncertainty has proven to be advantageous. In particular, a fluctuation around a nominal “starting value” of a parameter can be (partially) automatically impressed onto these parameters. The magnitude and type of the fluctuations used for this purpose are preferably oriented on the basis of the reality or the “expected reality”. Therefore, for instance, several irradiation plans can be calculated and they can be subsequently compared to each other. The calculation can be carried out in such a way that an irradiation plan is calculated for the case that the appertaining parameter has its nominal value, an irradiation plan is calculated for the case that the appertaining parameter has it typical maximum value, an irradiation plan is calculated for the case that the appertaining parameter has the maximum value that is to be expected in actual operation, an irradiation plan is calculated for the case that the appertaining parameter has acquired its typical minimum value, and/or an irradiation plan is calculated for the case that the appertaining parameter has acquired the minimum value that is to be expected in actual operation. In addition or as an alternative, it is also possible for (additional) intermediate values to be calculated. These can be selected, for example, in such a way that they are statistically distributed appropriately, for instance, in such a way that they correspond to the parameter values that are realistically to be expected over the course of time (preferably a suitable statistical weighting can be provided here). If several parameters are present, it is fundamentally possible in any desired way for the parameters in question to each be varied “one-dimensionally”, or for a variation of n parameters to be carried out in the form of an n-dimensional space. Of course, strategies that lie between these two extremes are also possible and, under certain circumstances, practical and preferable. Each of the irradiation plans obtained in this manner can subsequently be compared to each other. For example, it is possible for each of the obtained irradiation plans to be displayed “merely” to the person drawing up the irradiation plan. It is also possible that, through the use of mathematical fit methods, certain tendencies are displayed and/or, at least to a limited extent, an automated optimization is carried out. A calculation should especially be made for those parameters which, for example, experience has shown to have a particularly great influence on the result of the irradiation planning In contrast, however, parameters which experience has shown to have only little or (virtually) no influence on the irradiation plan should not be taken into account at all or only with a smaller “resolution” (calculation point density) for reasons relating to the computing time. It can be especially advantageous if the density of computation points for an individual parameter reflects its effect on the irradiation plan (for example, the effect to be expected on the basis of experience).

Fundamentally, all values that have an effect or influence on the irradiation planning can be used as an uncertainty and/or as a parameter, especially those that have a non-negligible effect, a substantial effect and/or a significant effect on the irradiation planning It is especially preferable if at least one uncertainty and/or at least one fluctuation of at least one parameter and/or at least one parameter, at least at times and/or at least partially, is taken from the group that includes the patient positioning, the movement detection, the beam range, the beam profile, the beam position and the type of tissue. It has been found that especially the above-mentioned quantities have a normally very pronounced effect on the irradiation planning The term “patient positioning” refers especially to positioning imprecisions of the patient. Typically, patients are positioned by means of an immobilization system or a patient positioning system, whereby positioning imprecisions that are typically in the range of millimeters can occur. The patient positioning can be dealt with during the dose calculation, for example, in the form of a movement of the isocenter and/or as a rotation of the beam inlet channel. The term “movement detection” especially refers to a quantity that occurs due to deviations during the detection of the movement of the patient or of parts of the patient. For example, the breathing of a patient can be tracked using strain gauges, imaging techniques (e.g. CT and/or monitoring with a video camera), and on this basis, conclusions can be drawn about the momentary position of a moving target volume area (e.g. a tumor in the lung tissue). Here, uncertainties can occur, which can be caused, for example, by detection errors of the measuring device (e.g. image errors of a video camera, measuring errors of a strain gauge, etc.), by errors in the correlation between the measured value and the position of the target volume area, by phase errors, by latency errors between the movement surrogate and the actual movement, and the like. Such errors can be dealt with, for example, by manipulations of the movement trajectory of the target volume area within the scope of a 4-D dose calculation. The term “beam profile” (lateral as well as longitudinal) refers especially to inadequacies in terms of the shape of the particle beam (as a rule, one should strive for a circular beam profile shape with a Gaussian profile) owing to technical limitations or inadequacies. The term “beam position” (lateral as well as longitudinal) refers especially to positioning errors that can occur due to a particle energy modulation device, due to errors of a lateral particle deflection system (for example, magnetic field coils) and the like. Such imprecisions can especially arise due to technical limitations or inadequacies. They can be dealt with by varying the isocenter and/or by rotating the beam inlet channel. The term “beam range” can especially refer to the range of the particle beam due to the different damping effect of different types of tissue in the patient. The so-called Hounsfield units, which can be read out, for example, from a CT data record, have to be converted into water-equivalent ranges for purposes of actuating a particle accelerator. This can be done, for example, using a table. However, such a table has only a finite precision. Uncertainties in the beam range can occur due to a manipulation of the Hounsfield unit range table and/or due to a global movement. The term “tissue type” especially refers to a value that takes into account uncertainties in terms of the (measured) tissue type, and thus in terms of the different damping effect and/or biological effectiveness of the particle beam on the tissue in question. This can be dealt with by varying the tissue boundaries and/or the tissue properties.

Advantageously, the method can be carried out in such a way that the effects of at least one uncertainty are calculated, displayed and/or taken into consideration, at least at times and/or at least partially, by comparing at least two, preferably a plurality, of irradiation plan results. In particular, comparing a plurality of irradiation plan results and/or taking into consideration two or more uncertainties can especially preferably be carried out (at least partially) automatically. In this context as well, of course, a (partial) manual user intervention and/or a manual user adjustment is conceivable. In particular, irradiation plan results can be used here that were determined by a variation at least at times and/or at least partially or by a fluctuation of at least one parameter. The obtained irradiation planning results (preferably determined on the basis of the fluctuation of at least one parameter, but optionally in another manner) can—as already explained above—be displayed “merely” to the person drawing up the irradiation plan and/or automatically, for example, by using generally known numerical optimization strategies, so as to ultimately arrive at an improved, especially more robust irradiation plan.

It is especially advantageous if, at least at times and/or at least in certain areas, a plurality of uncertainties is calculated, assessed, displayed and/or taken into account. Preferably, the uncertainties (or their consequences) taken into account are those that have substantial, relevant, significant and/or non-negligible effects on the irradiation planning It is particularly advantageous if (essentially) all such relevant parameters are taken into account. However, it can already prove to be advantageous if only one single uncertainty and/or a specific number (especially a partial set) of uncertainties is taken into account.

In particular, it is proposed to carry out the method in such a way that the effects of at least one uncertainty are displayed visually, especially graphically, at least at times and/or at least partially. It has been found that the human eye is particularly well-suited to process a large number of graphically displayed items of information within a short period of time. In this manner, the person drawing up the irradiation plan can use the method very conveniently, quickly and, as a rule, intuitively. Moreover, results of the irradiation planning that are typically very good can be achieved. Furthermore, it should be pointed out that already with today's method for drawing up irradiation plans, there is often a visual interface for the person drawing up the irradiation plans. Hence, the method can be carried out using existing hardware (or any hardware modifications can be kept to a small, feasible level) and/or the person drawing up the irradiation plan does not have to be extensively retrained before being able to use the method.

It can be advantageous if the method is carried out in such a way that, at least at times and/or at least partially, the effects of at least one uncertainty are output as an absolute value, as an absolute fluctuation, as a relative fluctuation, as a limit value approximation and/or as a flag display. A display as an absolute value can represent, for example, a calculated maximum value or minimum value (output, for example, as indication of the deposited dose). Moreover, a display in the form of a relative fluctuation is also possible, for instance, in that the display indicates by how many percent the value has exceeded or fallen below the “actual” dose to be deposited. An absolute fluctuation can also be indicated that represents, in units, whether the deposited dose has potentially exceeded or fallen below the desired dose (target dose). Another form of display is the extent to which a limit value is being approached or the extent to which it has already been exceeded (for example, in the form of a relative and/or absolute display). A flag display is also conceivable that indicates, for example, in binary form, whether the value is still within a permissible fluctuation range (or within a very narrowly selected test fluctuation range), or whether the value has already left this range. It is especially advantageous if the type of display can be changed and/or if one can switch over between different display modalities. It can also be advantageous if the change or the modification can be carried out by the person who is drawing up the irradiation plan. In particular, preliminary tests have shown that the use of several display modalities normally yields good results of the irradiation planning In particular, different display modalities are often desired or useful at different points in time when an irradiation plan is being drawn up.

It can be particularly advantageous if, within the scope of the method, at least at times and/or at least partially, a flicker display, a color-coded display, a grayscale display, an isoline display, a washing display and/or a symbol display are used. In particular it is advantageous if the type of display can be changed and/or modified, especially as a function of the specific wish of the person drawing up the irradiation plan. Here, too, especially the use of several display modalities can normally translate into a particularly high user convenience and/or a particularly advantageous irradiation plan. A symbol display can function, for instance, by displaying numerical values or else by displaying an “X” (for “lies outside of an additional limit value”) or a “checkmark” (for “lies within an additional limit value”). As a rule, color-coded displays, grayscale displays, isoline displays and washing displays are very intuitive for the person drawing up the irradiation plan. In particular, such displays are at times already used for drawing up irradiation plans, so that the proposed method can be learned very quickly. In particular, a flicker display is highly advantageous since different images are displayed consecutively in a time sequence. Here, for example, the additional dimension that is to be displayed can be indicated by the “time axis”. The flicker display is especially advantageous together with the other explicitly proposed display modalities, but also with all kinds of other display modalities. With the flicker display, the frequency of the image change can be selected in such a way that the change can still be perceived by the human eye. However, it is also possible to select the frequency of the image change to be so high that the image change is no longer perceived as such but rather, that the different images form one single image with “mixed colors” as seen by the human eye.

Another preferred refinement of the method is obtained when, at least at times and/or at least partially, the irradiation planning is carried out in the form of a 3-D irradiation plan and/or in the form of a 4-D irradiation plan. In this context, a 3-D irradiation plan is especially well-suited for essentially stationary target volume areas (if applicable, also for movable target volume areas that are being irradiated using “gating” irradiation methods). A 4-D irradiation plan is particularly advantageous when a moving target volume area is to be irradiated, especially when the moving target volume area is being actively “tracked”, which is done especially by means of so-called “tracking” irradiation methods (usually as scanning methods, spot-scanning methods, continuous scanning methods, raster scanning methods and/or intensity-modulated raster scanning methods).

Moreover, a device for drawing up an irradiation plan is being proposed that is configured and designed in such a way that it carries out a method with the properties described above. In an analogous manner, the device in question then has the above-mentioned properties and advantages. The device can be especially a “classic”, software-controlled electronic computer. Of course, the computers can consist of a plurality of individual computers that are linked via electronic networks. In any desired manner, these can be so-called workstation farms or distributed computer networks in which the computers are not located at a single site, but rather can be located physically far away from each other and can be linked together, for example, via the Internet, via virtual private networks (VPN) and the like (for example, so-called “distributed computing”). In particular, it is possible for the method to be carried out on the kind of devices that are already being used for drawing up “classic” irradiation plans. This permits a particularly quick use of the proposed method or a particularly quick migration to the proposed method.

Finally, a memory unit is also utilized that contains at least one irradiation plan that was at least at times and/or at least partially drawn up on the basis of the method described above. The memory unit can be any type of electronic memory unit such as, for example, the memory sector of an electronic computer (RAM, hard drives and the like). In particular, these can be any desired data storage media, such as, for instance, a state-of-the-art diskette, CD, DVD, blue-ray disc, USB stick, exchangeable disk, magneto-optical data medium, and the like.

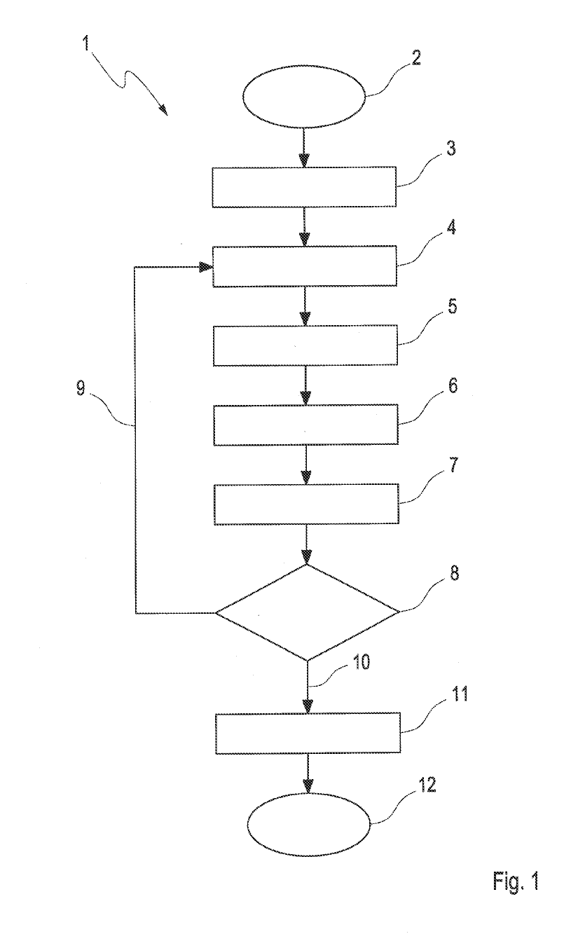

FIG. 1 shows a schematic flow diagram of a method for drawing up an irradiation plan 1, in which the effects of uncertainties on the irradiation result are taken into account within the scope of the irradiation planning

The method for drawing up an irradiation plan 1 begins with the starting step 2. The initial data for drawing up an irradiation plan is made available here. For example, data about the location, position, size, tissue type and the like of a tumor that is to be treated is read in as the initial data. Moreover, information is made available about the surrounding tissue and its radiation resistance, especially information about critical tissue that reacts particularly sensitively to a higher exposure to a dose (so-called OARs, short for “organ at risk”). Furthermore, the target dose distribution prescribed by the physician is on hand during the starting step 2 of the method 1. This prescription defines, for example, the radiation load that is to be applied to the tumor tissue. Optionally, information about a maximum dose for (parts of) the surrounding tissue is provided.

Based on the information made available in the starting step 2, the tumor, the risk structures and, if applicable, other tissue regions are constructed in the subsequent step 3. That is to say, the location and size of the tumor and of the risk structures are converted into the “numerical format” of the device on which the irradiation plan is being drawn up (for example, a high-performance computer). Thus, for instance, the appertaining tissue regions can be displayed with delimitation lines, in a manner that is intuitively clear.

Now all of the data is available to draw up and optimize an initial irradiation plan in the subsequent step 4. In this process, the initial irradiation plan is drawn up or optimized with nominal parameters. In other words, to start with, it is assumed that all of the input data such as, for instance, the information about the position of the tissue in question, is completely correct, that is to say, that no measuring errors or other changes have occurred. By the same token, it is assumed that all of the machine parameters and the like are error-free, so that, in particular, no beam positioning errors, beam energy errors, beam shape errors and the like can occur. This matches the prior-art irradiation planning (disregarding the “feel” of the person drawing up the irradiation plan). Merely for the sake of completeness, it should be pointed out that, as a rule, the irradiation planning is carried out iteratively and, at times, several initial attempts by the person drawing up the irradiation plan might be needed (attempts that are conceivably started with manual specifications drawn up on the basis of the person's “feel”).

It is easy to see that the assumption of ideal data is not always accurate in actual practice. In actual practice, all of the initial data (for example, the location of the tumor tissue) is always associated with a certain degree of error. On the one hand, these errors can be due to the measuring equipment (for example, in conjunction with the detection using a computer tomograph (CT) or some other detection system). In particular with 4-D irradiation methods (that is to say, with methods for irradiating moving tumors), it is impracticable or undesirable to use a CT during the irradiation. Therefore, in such cases, a so-called movement surrogate is normally obtained with a CT simultaneously with the data acquisition. This can be an acquisition of movement with a video camera, a strain gauge placed around the chest, or the like. Subsequently, during the actual therapy, information can be obtained from the movement surrogate about the CT data and thus about the actual position of the target volume area that is to be treated. However, it is also possible that an error of a non-technical nature is made. For example, several hours and/or days (that are used, for instance, for drawing up the irradiation plan) can lie between the CT measurement and the actual therapy. During this period of time, biological effects can lead to a location change, density change and/or size change of the tumor tissue. This also causes errors that cannot be (completely) controlled. Other errors can arise because of the device itself Thus, due to technical limit values, the generated particle beam cannot be totally precise, as a result of which, for example, deviations can readily occur in the particle energy, particle position and particle geometry. Fundamentally, the errors can be relatively small, but in spite of their conceivably slight discrepancy from the target value, they can have quite significant effects on the irradiation plan. Thus, especially in the area of tissue transitions and/or in specific tissue regions, it is very well possible that unacceptable changes in the ultimately applied dose might occur.

In order to check the robustness of the irradiation plan calculated and optimized in step 4, in the proposed method 1, another step 5 is carried out in which a plurality of (relevant) parameters is varied. The result is that, with a number n of parameters, an n-dimensional parameter space is created. For each parameter set in the n-dimensional parameter space, the resulting dose distribution per parameter set is calculated here. The variation of the (plurality of) (relevant) parameters is carried out automatically in the present embodiment. The scope of the variations is determined, for example, by the parameters of the irradiation device for which the irradiation plan is being calculated, by the tissue distribution in the patient to be treated, etc. The appertaining values can (also) be read in within the scope of starting step 2. Of course, it is possible that, when the irradiation plan is being drawn up, manual user intervention can be taken in terms of varying the parameters. This especially also includes different calculation patterns and/or the use of different calculation algorithms (whereby the calculation in question can, once again, be carried out largely automatically).

An example of parameters that are changed in the embodiment shown (whereby it is possible to leave out certain parameters and/or to take into account additional parameters) is the precision of the patient positioning that can be achieved by the immobilization system or patient positioning system employed. An imprecision in the positioning of the patient can be taken into account by moving the isocenter of the applied particle beam and/or by rotating the beam inlet channel. Another parameter that can be taken into consideration (especially in the case of 4-D irradiation methods) is the movement detection, which can be taken into account, for example, when a movement surrogate is used. During the movement detection, imprecise measured values can be present due to imprecise amplitudes, imprecise phases and/or a latency between the movement surrogate and the actual movement (that is to say, a type of phase shift). These imprecisions can be simulated during the calculation by means of suitable manipulations of the movement trajectory of the target volume area used for the 4-D dose calculation. An example of another parameter is the beam range. The starting point for the irradiation plan is a 3-D data record or a 4-D data record. The “coloration” (tissue intensity) that appears in the CT data record does not correspond to the water-equivalent range as is “seen” by the particle beam. A conversion of the “CT data” (measured in Hounsfield units—HU) into the water-equivalent range is carried out on the basis of appropriate conversion tables as well as on the basis of the parameters of the direction of the irradiation. Since such a table only has a finite precision (but usually for other reasons as well), corresponding uncertainties in the beam range normally occur. These uncertainties can be taken into account during the present calculation by means of a manipulation of the Hounsfield unit range table or by a global movement. Another example is an uncertainty in the beam profile (lateral and longitudinal) that can occur due to technical limits or inadequacies during the acceleration process or beam guidance process. The corresponding uncertainty can be taken into account through an appropriately modified physical dose application per tissue volume unit (raster point). Another example is the uncertainty of the biological model that was used to draw up the irradiation plan. Such uncertainties can be dealt with through modified biological model parameters.

The variation of the parameters in method step 5 is advantageously carried out in such a way that a certain number of intermediate points are taken into account. The density of the intermediate points can especially be increased in those areas where the resulting dose distribution changes especially markedly (thus, where the effects of the parameter fluctuations are very pronounced). This increases the probability that the local maxima or the local minima will be detected as completely as possible. The variation of the parameters should also be carried out in a range that is selected in such a way as to cover all of the typically occurring parameter changes and/or all of the maximum parameter variations that can be expected during actual operation. It can also be useful that, in addition to the above-mentioned values, a certain safety margin is still added so that, for instance, another 50% is calculated above the maximum parameter fluctuation that can be expected during actual operation (based on the distance between the nominal value and the maximum fluctuation value that can be expected during operation).

Since it could be the case that a larger number of parameters and parameter variations has to be calculated, the method step 5 can require a longer calculation time. In particular, it might be necessary to calculate several hundred or several thousand dose distributions.

In the subsequent step 6, the dose uncertainties or other statistical fluctuations per volume unit are determined. These uncertainties can be stored in a suitable format such as, for example, in an appropriate dimensional matrix. For example, in this step, absolute deviations from the target dose, relative deviations from the target dose, absolute applied doses, binary data (that indicate, for example, if a dose is still within a permissible dose interval or not), and the like can be calculated and stored. Moreover, it is possible that more in-depth calculations are carried out, especially summing operations and integration operations. Such calculations are especially useful (and as a rule, to be carried out at a certain—even if later—point in time), if histograms and the like are to be displayed. In this context, it should be pointed out that it is precisely medical personnel that likes to make use of so-called “dose histograms” within the scope of checking an irradiation plan. Accordingly, a higher level of acceptance on the part of the medical personnel can be attained if such dose-volume histograms can also be generated within the scope of the “error assessment display” being proposed here.

Subsequently, in method step 7, the dose variation (dose uncertainty) is displayed. This can be done, for example, in that the nominal dose distribution (target dose distribution) is displayed so as to be superimposed with an uncertainty distribution. The display can be, for example, a so-called flicker plot in which the nominal dose distribution and the uncertainty distribution are displayed at a relatively high frequency consecutively and alternating. Experience has shown that the eye reacts relatively sensitively to movements so that a person can perform a good qualitative and/or quantitative analysis using such a flicker plot.

In addition or as an alternative to the nominal dose distribution (especially alternating with a nominal dose distribution), for example, the maximum dose and/or the minimum dose on the basis of the uncertainty analysis (method step 6) can be displayed. By the same token, in addition or as an alternative, a binary data record can be displayed that indicates, for instance, in green or in red, whether a prescribed acceptance interval has been reached. Likewise, in addition or as an alternative, a distribution that quantifies the uncertainties (for example, a confidence interval) can also be displayed so as to flicker in complementary colors. The uncertainties can especially be scaled in such a way that their colors resemble the dose values of each of the individual volume areas when the uncertainty is small and/or can be tolerated, or else they are displayed as complementary colors. These voxels can then appear gray (in particular in case of a high-frequency flicker). By the same token, instead of a flicker, the distribution can be displayed with a certain transparency (for example, 50% transparency) statically superimposed over the nominal distribution (for example, using colors that are complementary to the nominal distribution). The transparency can ensure that dose values with small uncertainties are displayed, for example, in a grayscale. In contrast, larger deviations can be displayed so as to be emphasized by color (in the flicker display as well as in a transparent or other display). Here, the color can serve as a measure of the deviation.

Another possibility is for each volume area in the displayed images (especially sectional images) to be displayed with a superimposed symbol that indicates whether a confidence interval is being adhered to. For example, a “checkmark” can indicate that the uncertainty lies within a tolerable interval, whereas an “X” indicates that the limit has been exceeded. A quantitative display is also possible here, for example, in that more or fewer rectangular frames are displayed (histogram-like display).

Moreover, a display as a contour plot is also possible. In particular, a display can be superimposed over the CT data. Here, it is especially possible that, on the basis of the “truly visible structure”, an especially intuitive qualitative and/or quantitative assessment can be made by the person drawing up the irradiation plan.

Another display possibility is based on the dose-volume histograms often used nowadays particularly by medical personnel. Thus, the uncertainties that occur can be displayed in the form of error bars that are superimposed over dose-volume histograms. Of course, a display that uses grayscale shading and/or colors and/or some other technique is also conceivable.

Based on the display generated in step 7, during the subsequent step 8, the quality and especially the robustness of the irradiation plan drawn up within the scope of method 1 (until now) is assessed. Depending on whether the quality and/or robustness of the irradiation plan has been assessed as being adequate, the process either jumps back(=step 9) to method step 4 or jumps forward(=step 10) to the next method step 11. In method step 11, the generated irradiation plan is stored, for example, on a data medium (DVD, CD and the like). Thus, the method 1 ends with step 12.

Of course, it is possible that the assessment 8 is not performed (exclusively) by one person. Rather, it is possible that, for instance, in addition or as an alternative, an automatic assessment procedure is carried out.

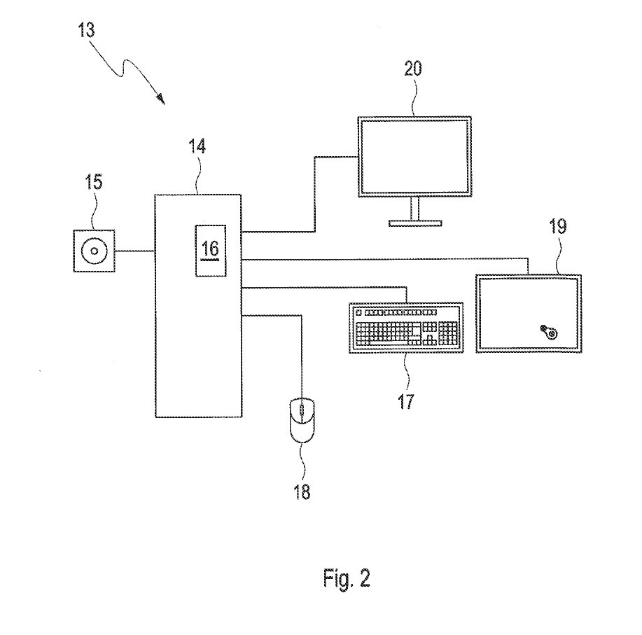

FIG. 2 shows a schematic view of a planning device 13 on which, for example, the method 1 shown in FIG. 1 for drawing up an irradiation plan can be carried out. The planning device 13 is based on a program-controlled electronic computer 14. In order to increase the computing capacity of the computer 14, it can have several processors and/or be configured as a so-called cluster. The computer 14 has an internal memory 16 (for example, a hard drive) on which an appropriate program code that executes the method 1 is stored. Here, it is very well possible for the program code stored in the internal memory 16 to be loaded, for example, in a volatile working memory (so-called RAM) in order to be executed.

Moreover, the computer 14 has a data input/output unit that, in the embodiment shown here, is configured as a DVD drive 15. Via the DVD drive 15, for instance, patient data, machine parameters, a prescribed dose distribution and the like can be read into the computer 14. Likewise, via the DVD drive 15, the finished irradiation plan can be output and stored. The DVD drive 15 can be, for example, a commercially available DVD burner that can not only read out data from CDs or DVDs, but that can also write data onto blank CDs or blank DVDs. Of course, it is also possible to provide a plurality of DVD drives 15.

The computer 14 is operated by means of generally known data input units such as, for example, a keyboard 17, a mouse 18 and/or an electronic drawing board 19. In the present case, the irradiation plan as well as its uncertainties are output via one or more monitors 20.

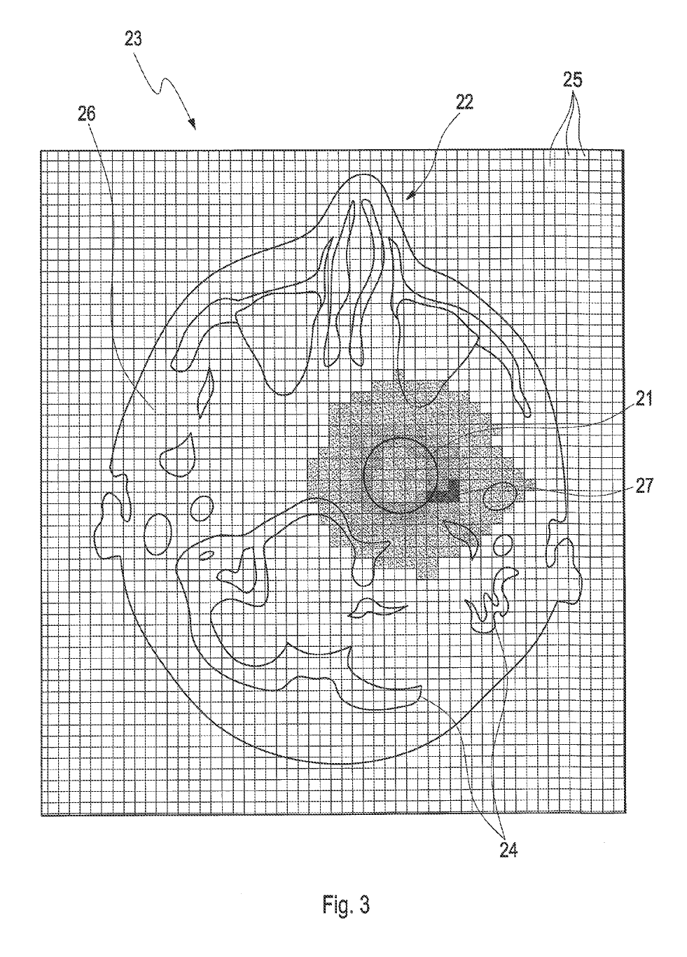

FIG. 3 shows a first example of a data output that was generated using a method 1 for drawing up an irradiation plan according to FIG. 1 (or according to another embodiment of an irradiation plan).

Here, by way of example, a tumor region 21 to be treated and located inside the head 22 of a patient (brain tumor) is selected. As usual, the tumor region 21 (which is optionally surrounded by a certain, fairly small safety margin) is to receive a radiation dose so that the tissue cells located in the tumor region 21 are severely damaged or killed off. In contrast, the tissue outside of the tumor region 21 should be exposed to as little radiation as possible or to no radiation at all. In the embodiment shown, the tumor region 21 is drawn as a circle. In actual practice, it will generally have different shapes; however, the exact shape of the tumor region 21 is inconsequential in order to explain the present embodiment. Moreover, in the display 23, tissue contour lines 24 are drawn that serve to orient the user of the planning device 13—and thus to facilitate the work being done. The display 23 can be shown, for example, through an appropriate selection on the monitor 20 of a planning device 13 and, if applicable, it can be varied.

In the display shown in FIG. 3, a fluctuation (change) in the dose distribution is calculated by varying input parameters within the scope of drawing up an irradiation plan (see FIG. 1), and displayed in the form of different grayscales. Here, within the scope of the calculation of these dose fluctuations, a certain grid 25 with a certain precision (grid resolution) was selected, whereby the grid 25 can be recognized in the form of fine lines in FIG. 3. The resolution of the grid 25 can, of course, be selected to be finer or coarser. Moreover, one can also visualize different grid resolutions in different spatial directions and/or different grid resolutions in different sectors of the display 23 (for example, a finer grid resolution in a volume area in or adjacent to the tumor region 21).

As is generally the case with an actual irradiation, during the calculation, a fluctuation of input parameters (for example, of device parameters and the like) in regions 26 that are far away from the tumor region 21 does not lead to any change (or at most, to a minimal change) in the dose that is deposited in the tissue regions 26 in question. Accordingly, no (perceptible) gray coloration can be seen in these far-away tissue regions 26.

However, if one reaches regions that are adjacent to the tumor region 21, the gray shading increases markedly, which can be seen very clearly in FIG. 3. The stronger the gray shading, the more strongly the deposited dose fluctuates when the input parameters change.

In the embodiment shown in FIG. 3, the fluctuation in most of the tissue regions of the head 22 fall within a very acceptable fluctuation range. The grayscales are only slightly shaded. A somewhat different situation applies to the problem region 27, which can be seen in FIG. 3, where a variation of input parameters leads to a substantial change in the deposited dose. For this reason, the problem region 27 is filled with a very strong gray shading. For the user of the planning device 13, this is a sign that he/she should draw up a new irradiation plan that does not bring about such a strong dose variation in the entire head region 22 when the parameter values are changed. In other words, the user of the planning device 13 will try to calculate an irradiation plan in which the display 23 of dose variations only shows raster points with a slight gray shading over the entire region. This especially applies if a (particularly) critical tissue region is located in the problem region 27 (for example, a brain region with an important function and/or with a blood vessel). In such a case, if applicable, then one can consider an irradiation plan to be acceptable if there is a problem region 27, but it is located outside of this (and other) critical tissue regions. As can be seen in FIG. 3, no critical tissue region is present in the problem region 27 shown there.

In order to further increase the convenience for the user of the planning device 13, it is, of course, also possible to additionally or alternatively use a color scale instead of a grayscale.

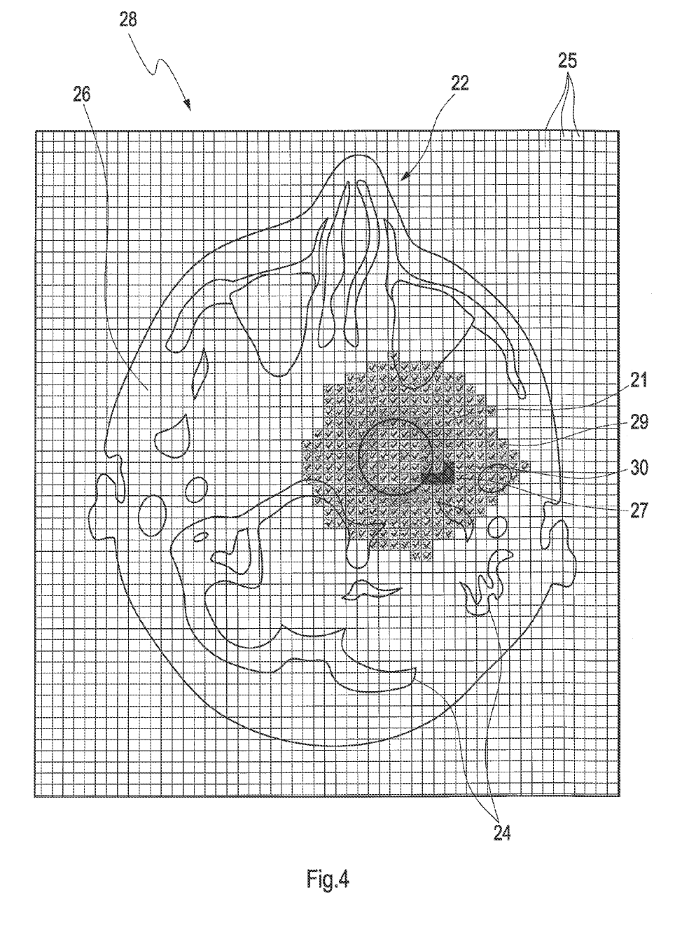

A refinement of the display 23 of dose fluctuations shown in FIG. 3 is the display 28 of dose fluctuations shown in FIG. 4. As can be seen, the display 28 shown in FIG. 4 is quite similar to the display 23 shown in FIG. 3. However, as an additional aid to the user of the planning device 13, there are also flag values in the form of a checkmark 29 or an “X” 30. In this context, a checkmark 29 means that a maximum dose fluctuation prescribed by the physician is not exceeded. Thus, for example, the value neither exceeds nor falls below a maximum dose specified by a physician for a given tissue region or a minimum dose specified by a physician for a given tissue region, so that in this manner, overdoses or underdoses can be avoided. Accordingly, an “X” 30 indicates that an excessive fluctuation of the entered dose has occurred, which the physician has deemed to be impermissible. Accordingly, the display 28 of the dose fluctuations shown in FIG. 4 has to be rejected because of the “Xs” in the problem region 27.

In order not to overload the display 28 for the user of the planning device 13, no flag display has been implemented in tissue regions (especially in far-away tissue regions 26) where the dose fluctuation turns out to be particularly small. There, neither “Xs” 29 nor checkmarks 30 are displayed. This not only simplifies the overview, but also provides the user with a sort of “third flag” for an especially low dose fluctuation.

Moreover, it is also possible that the fluctuation values that the physician has deemed to be permissible can be “made more stringent” by the user of the planning device 13 through user inputs to this effect. In this manner, the user of the planning device 13 can draw up a particularly robust irradiation plan in an especially simple and convenient manner.

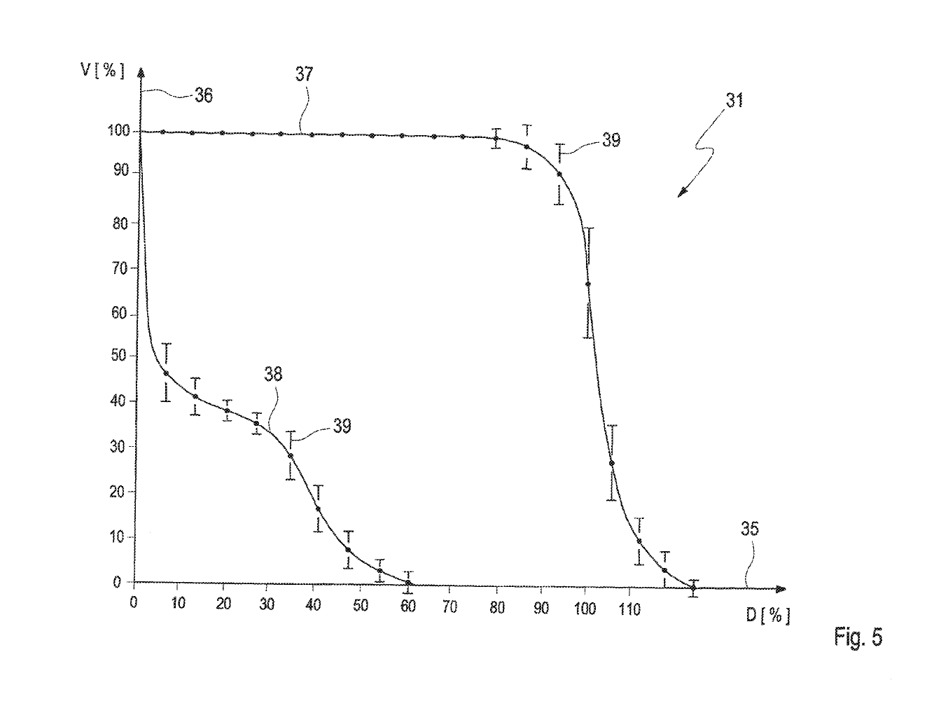

FIGS. 5 to 8 show further displays 31, 32, 33, 34 of dose fluctuations. Here, the displays 31, 32, 33, 34 are based on so-called dose-volume histograms of the kind currently being used for radiation purposes (and which are particularly appreciated by medical personnel). The displays 31, 32, 33, 34 each show the dose (in percent) along the abscissa 35, while the volume (likewise in percent) is shown along the ordinate 36.

The display 31 (FIG. 5) shows the appropriate dose-volume curve 37 for the target volume CTV (Clinical Target Volume) as well as the dose-volume curve 38 for critical tissue regions OAR (Organ At Risk). Aside from the actual curves 37, 38, error bars 39 have also been plotted that indicate the change of each individual curve 37, 38 as a function of fluctuations of the input parameters. The precise definition of the error bar 39 shown can vary (for example, as a function of special needs of the user). Thus, error bars 39 can indicate, for example, a 5% to 95% interval. Of course, other interval limits or other meanings are also conceivable.

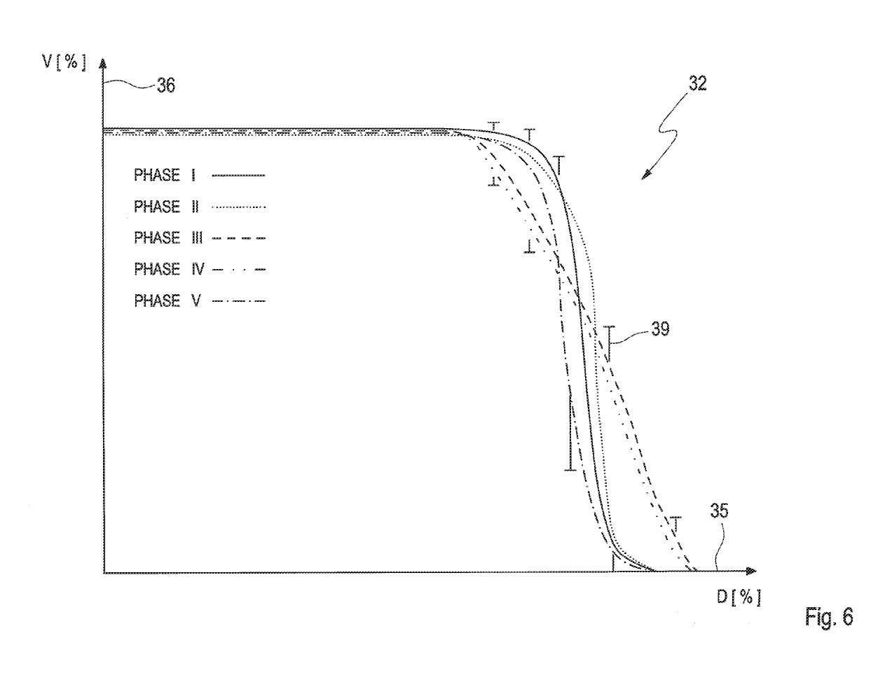

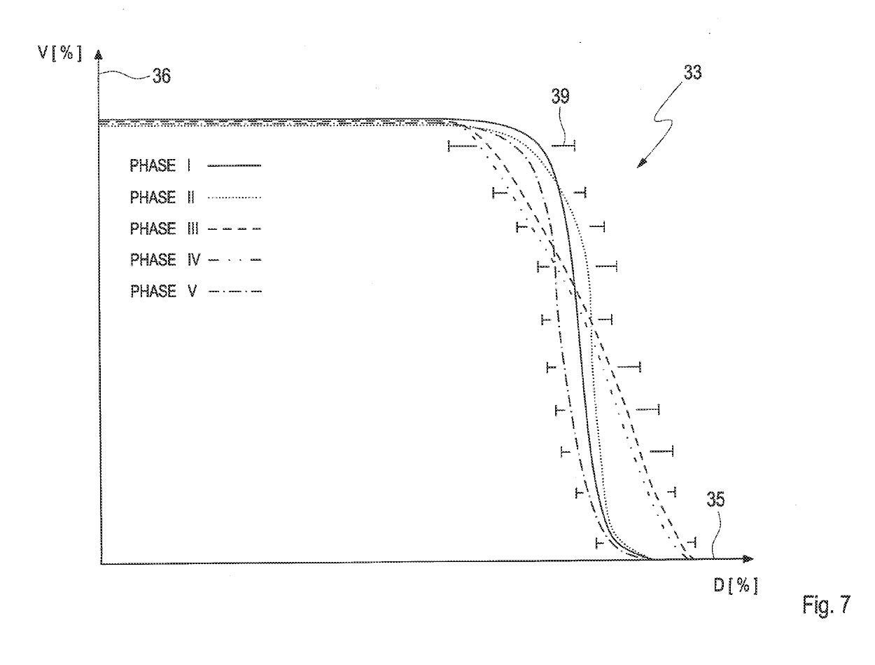

FIG. 6 shows a display 32 that has been changed as compared to that of FIG. 5. The present display 32 shows the situation for several different phases I, II, III, IV and V (each drawn with different types of lines). In this manner, an especially advantageous assessment of the robustness of the irradiation can be carried out, particularly in the case of moving target volumes (4-D irradiation method). The error bars 39 shown can be displayed “cumulatively” for the various phases, or else they can be displayed one at a time per individual phase I, II, III, IV and V. Of course, a change is also conceivable (for example, as a function of a user request). Moreover, the error bars 39 can be drawn in not only vertically but also, in addition or as an alternative, horizontally, which is shown in the display 33 of FIG. 7.

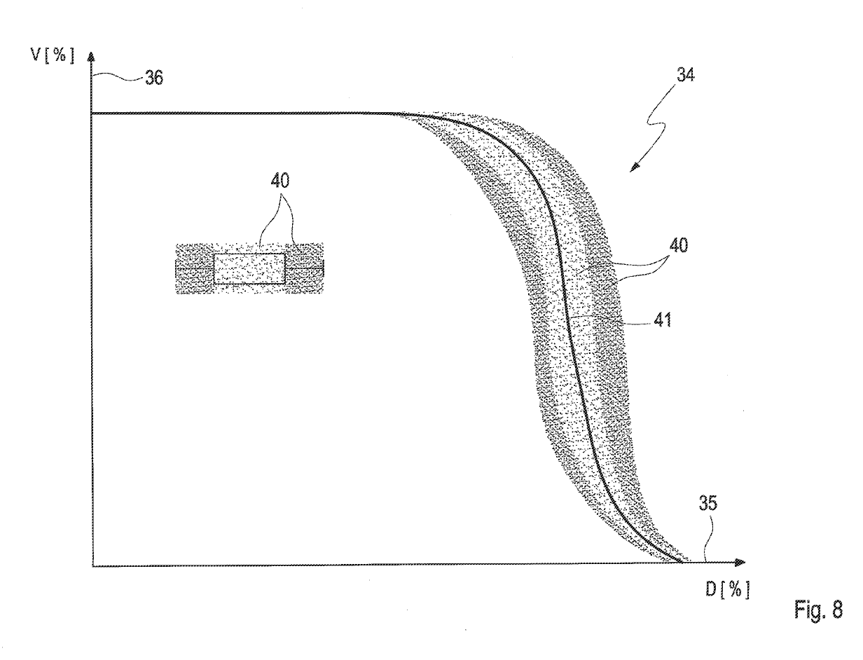

Finally, FIG. 8 shows another display possibility 34, which is based on dose-volume histograms. The display, which can be based on grayscales or colors 40 (whereby the grayscales or colors 40 are indicated by different cross-hatching 40 here), represent different interval limits for different “error bars” so that they can be detected simply and quickly. In addition to the various grayscales and/or colors 40, the display 34 shown in FIG. 8 also has a median line 41 drawn on it.

Merely for the sake of completeness, it should be pointed out that a dose-volume curve 38 for critical tissue regions can also be drawn in the displays 32, 33, 34 according to FIGS. 6 to 8 (similar to the display 31 in FIG. 5).

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B.” Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” should be interpreted as including any singular entity form the listed elements, e.g., A, any subset from the listed elements, e.g., A and B, or the entire list of elements A, B and C.

### LIST OF REFERENCE NUMERALS

1 method for drawing up an irradiation plan

2 starting step

3 construction of tissue structures

4 drawing up the irradiation plan

5 parameter variation and calculation of the dose distribution

6 determination of the dose uncertainty

7 display of the dose uncertainty

8 assessment

9 jump back

10 jump forward

11 storage of the irradiation plan

12 end of the method

13 planning device

14 computer

15 DVD drive

16 internal memory

17 keyboard

18 mouse

19 electronic drawing board

20 monitor

21 tumor region

22 head

23 display of dose fluctuations

24 tissue contour lines

25 grid

26 removed tissue regions

27 problem region

28 display of dose fluctuations

29 checkmark

30 “X”

31 display of dose fluctuations

32 display of dose fluctuations

33 display of dose fluctuations

34 display of dose fluctuations

35 abscissa

36 ordinate

37 dose-volume curve for target volume

38 dose-volume curve for critical tissue region

39 error bar

40 grayscale/color

41 median line

1-11. (canceled)

12. A method for drawing up an irradiation plan comprising at least one of calculating, assessing, displaying and taking into consideration effects of at least one uncertainty on the irradiation plan, at least one of at times and in certain areas.

13. The method according to claim 12, wherein the at least one uncertainty, at least at times and/or at least partially, constitutes a fluctuation of at least one parameter.

14. The method according to claim 13, wherein the fluctuation of at least one parameter is within a typical and/or maximally expected scope.

15. The method according to claim 12, wherein at least one uncertainty and/or at least one fluctuation of at least one parameter and/or at least one parameter, at least at times and/or at least partially, is taken from the group that comprises the patient positioning, the movement detection, the beam range, the beam profile, the beam position and the type of tissue.

16. The method according to claim 12, wherein the effects of at least one uncertainty are calculated, assessed, displayed and/or taken into consideration, at least at times and/or at least partially, by comparing at least two irradiation plan results.

17. The method according to claim 12, wherein, at least at times and/or at least in certain areas, a plurality of uncertainties is calculated, displayed and/or taken into consideration.

18. The method according to claim 12, wherein the effects of at least one uncertainty are displayed visually at least at times and/or at least partially.

19. The method according to claim 18, wherein the effects of at least one uncertainty are displayed graphically at least at times and/or at least partially.

20. The method according to claim 12, wherein, at least at times and/or at least partially, the effects of at least one uncertainty are output as an absolute value, as an absolute fluctuation, as a relative fluctuation, as a limit value approximation and/or as a flag display.

21. The method according to claim 18, wherein, at least at times and/or at least partially, a flicker display, a color-coded display, a grayscale display, an isoline display, a washing display and/or a symbol display are used to display the effects of at least one uncertainty.

22. The method according to claim 12, wherein, at least at times and/or at least partially, the irradiation planning is carried out in the form of a 3-D irradiation plan and/or in the form of a 4-D irradiation plan.

23. A device for drawing up an irradiation plan, wherein the device is configured and designed in such a way that it carries out a method according to claim 1.

24. A memory unit comprising instructions for at least one irradiation plan that, at least at times and/or at least partially, was drawn up by using a method according to claim 1.

25. The memory unit according to claim 24, wherein the memory unit is a data storage medium.
